(19) United States
(12) Patent Application Publication
(10) Pub. No.: US 2006/0290886 A1
(43) Pub. Date: Dec. 28, 2006

(54) Digital Capturing Pharmaceutical System

(75) Inventor: Dario Marcondes Santos, Dearborn, MI (US)

Correspondence Address: DARIO MARCONDES SANTOS, 4114 CALHOUN ST., APT. 404, DEARBORN, MI, 48126, US

(73) Assignee: Mr. Dario Marcondes Santos, Dearborn, MI (US)

(21) Appl. No.: 10/908,735

(22) Filed: May 24, 2005

(51) Int. Cl. A61B 3/02

(52) U.S. Cl. 351/243

(57) ABSTRACT

DCPS Digital Capture Pharmaceutical System is consisting of a brand new use for a widely existing specific available hardware, in the purpose of collecting pharmaceutical doctor/consumer/discount coupon/drug and drug sales information, done at pharmacies, drugstores or like. The developed system, that allows this new utility model, works on the Client Side and or Server Side.

This present New Utility Model improves the capturing of digital data from Pharmaceutical product sales on pharmacies or drugstores. This tech concept, marries the image from a doctor's prescription form plus the image of discount coupons to its sold drugs in the same session top-down routine, and records them into terminal's local database tables. The capturing of the prescription's image+coupon image and its information data from sold medicines are both generated in the same terminal set. In other words, the same program that captures the information data from sold drugs also captures the corresponding doctor's prescription+discount coupon images. This program is also capable to transfer electronically all captured information (each image set plus its transaction data set) to an output device or transmit them to a centralized remote site or to other computer or like. Presently, a number of market survey's companies working for the Pharmaceutical Industry, still use obsolete devices for capturing information from drug sales on Pharmacies, Drugstores or like. Most of those systems have an estate of technique that causes failures and delays between the time that the doctor's prescription is handed to the pharmacist/attendant until the moment that the sold product information, can arrive and be appended into a database in a central site. It happens especially in Latin America and Spain.

Why this entire process is necessary?

It is mandatory that a survey company has to proof for any pharmaceutical laboratory, that the general information collected from a specific sold product on the market, is really accurate. That's why surveys companies, need to keep the image of the doctor's prescription+coupon married to the record of the sold product as well. The obsolete image capturing techniques applied by surveys companies, includes loosed copying of prescriptions forms, loosed prescriptions faxing forms, loosed digital photos of the prescription forms sent over emails and etc. Of course, they did try in the past, to scan prescriptions forms on flatbed scanners, regarding the warming up time of the flatbed scanner turned this procedure impossible because the prescription must be released back to the consumer at the counter immediately. The last stage of their technology includes microfilming doctor's prescriptions forms in some pharmacies. The microfilm machines' manufactures are closing down and fortunately we found this brand new use for Lottery terminals.

Lottery Terminals are the Perfect Hardware for Capturing Pharmaceutical Retail Commerce Information.

Lottery Terminal manufactures, in order to speed up the reading of bet orders (game slips), developed front flip image scanners with a wide width input paper tray, married to a very fast paper tracking mechanism. These game slip lottery scanners are capable to read many different sizes of bet forms quickly. These scanners have a mark's identifier that allows the reading of the bet forms inserted face out in almost different positions. It is always ON, and ready-for-use. For instance, this engineering field defined the reading capturing area equal to the Letter size paper sheet. In addition, they made possible the scanned image available for capturing by programming. So this type of scanner is perfect to scan and keep the images of all doctor's prescriptions form sizes, at 200 DPI and many applications either Windows or Linux supported, can be applied on these captured image files, to compress them out, to a small size file which is easy to be transferred (6 Kb-40 Kb) through of any cheap low speed connection as for example, a 56 Kbs dial-up Internet connection. The majority of the Lottery terminals have the same desktop PC architecture, plus the front game slip image scanner, and the TFT LCD touch screen devices.

Utility Model Subject

DCPS Digital Capturing Pharmaceutical System—It's the new use of Lottery terminals or future assembled-like terminals, in the purpose of collecting pharmaceutical (doctor/consumer/drug/discount coupon) and sales information, done at pharmacies, drugstores or like. The new hardware set can include either its touch-screen device or not, also consider the use of the regular keyboard and mouse, both connected to the Lottery Terminal, from now on, known as Digital Capturing Pharmaceutical System's Terminal or simply DCPS terminal.

Hardware

The hardware solution found was to add and connect to the Lottery terminals: a Barcode reader device connected to any available port (USB, SERIAL or PS2), plus an external dial-up modem device connected to any available port (USB or SERIAL).

Software

Software works by the client side:

a) To implement the Linux Operational System on the Lottery terminals (Windows OS Family can be applied);

b) The development of the DCPS software—client side—Makes the terminal interact with the attendant (pharmacist or operator), though its displays, barcode reader, touch screen dialogs and its virtual keyboard, concentrate and transmit information digitalized data.

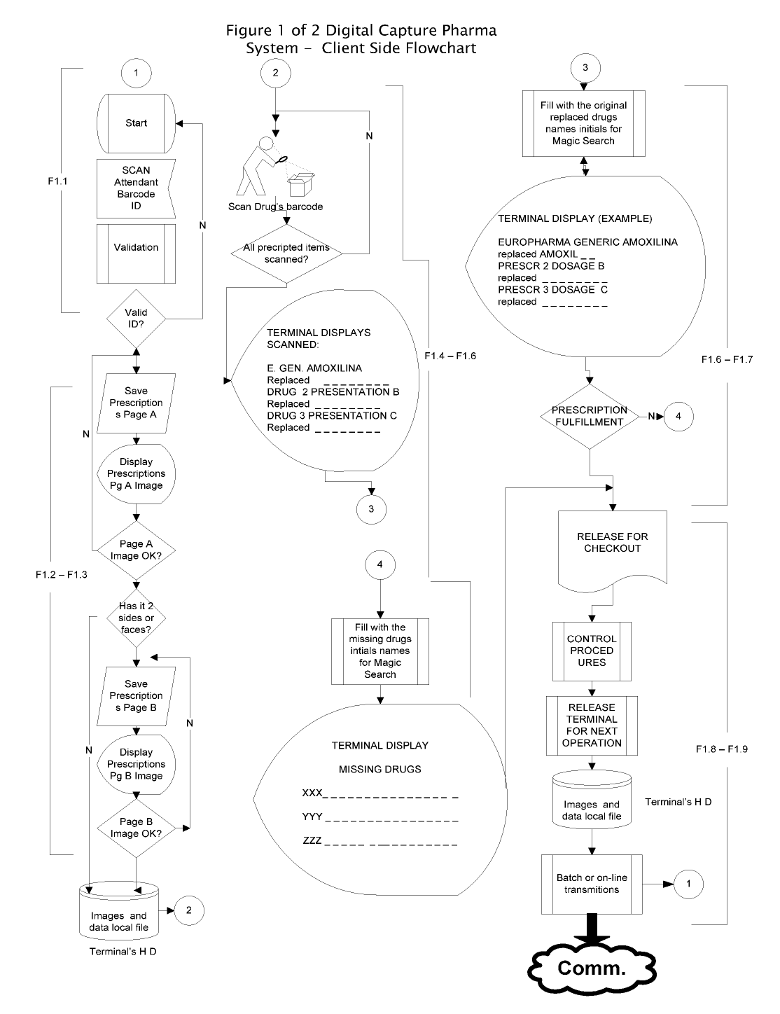

FIG. 1 (F1) Pharmacy/Drugstore routine flowchart running at DCPS terminals-client side.

F1.1 Program asks the operator's barcode id. The program will keep asking it until a valid operator's barcode ID be scanned by the barcode scanner.

F1.2 Program requests the operator to position correctly the doctor's prescription form on the front scanner tray, and then the operator tells the terminal to proceed to scan that prescription form. If the doctor wrote on both sides of prescription, then the operator will inform it to the program which will ask the operator to scan the prescription again with backside turned face out.

F1.3 Program asks the operator if the prescription's images are legible. If not, the operator should perform the item F1.2 again.

F1.4 Program asks the operator to either type doctor's code ID or highlight and selects it directly from the screen where the doctor prescriptions' images are shown.

F1.5 Program captures medicines codes one by one, through the shots upon their barcode labels printed on the drugs package (EAN barcode standard system or like);

F1.6 Program asks the operator if it is an original prescript drug or it is an original requested drug which was replaced for an equivalent one;

F1.7 Program asks the operator if there is any missing medicine which was impossible to substitute. The program captures information about missing medicine on the local pharmacy or drugstore when the operator uses a magic search on the drugs' missing box drug.

F1.8 Program saves the records of the prescription and coupon images from all captured and typed data onto the hard drive for a while. If the terminal has a communication facility connected full time, the record from each transaction, can be transferred on-line, otherwise, a cut off time should be defined and an accumulated set of records can be transferred on batch mode (one or more times a day) through any type of communication to a concentrator, to a server, to a remote computer, or to a remote datacenter etc.

F1.9 A full captured transaction, in the DCPS terminal, is mainly formed by: prescription's image, coupon's image (if any), “prescript medicine not available in the local stock”, “prescription's sold drugs list”, “prescription partially fulfilled”, “prescript medicine replaced by—”, “doctor's legal local registration numbers (CRM in Brazil)”, “doctor's clinic or hospital names”, “the patient name”, and or, “patient address”, and or, “patient proper ID” and or, “health insurance plan ID”, and or, “hospital or clinic ID”, and or, “point of sale address”, and or, “point of sale system code”, and or, “transaction date”, and or, “pharmacist/attendant identification”. The new program running on the DCPS terminal, also will be able to concentrate, manage and emulate from transmitted image files, the purchase process of drugs from prescriptions made over the phone, Fax and Internet (emails or proper forms). As result, the terminal will print and or display orders from consumers made remotely for delivering of prescript medicines to their places.

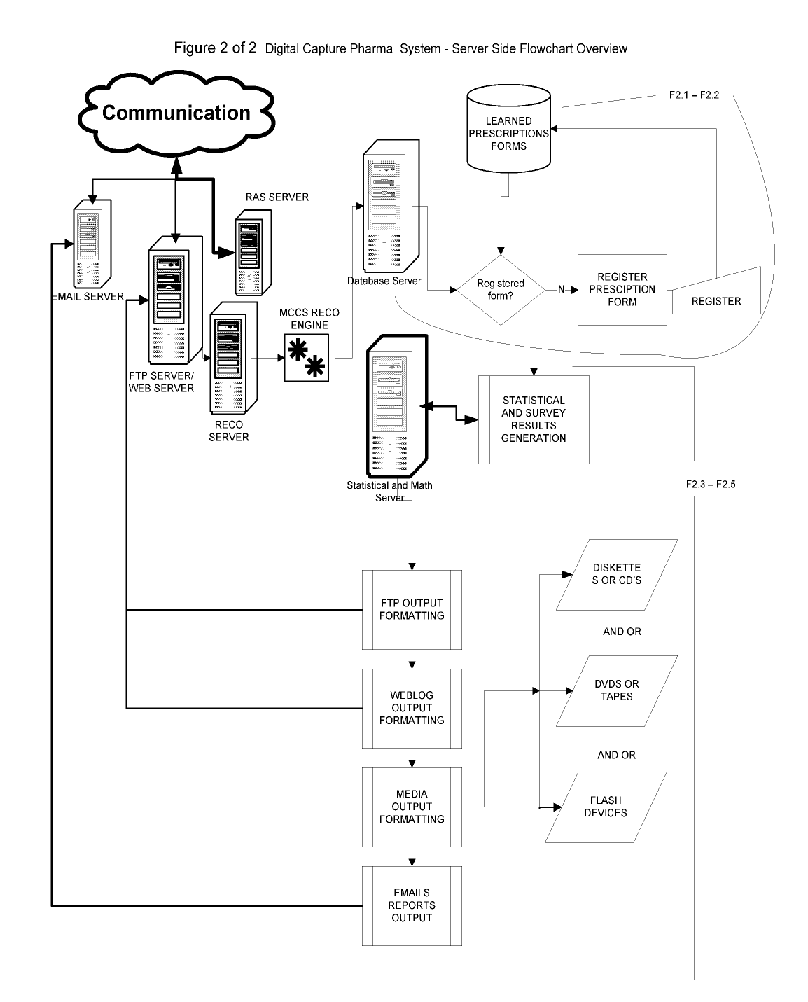

FIG. 2—DCPS System—Server side Overview Flowchart

F2.1 From the very first time that an unknown doctor's prescription is received on the server;

F2.2 it is redirected to the operator, who will register that prescription image into the pattern bank system, turning all logos, handwritings and typographic printing of that prescription, into a unique identifier set index pattern clues;

F2.3, this previous registering will allow, further links of that new doctor's profile based only on his own personal next prescription images.

F2.4 Once registered, every time that a doctor's prescription image inputs, the system automatically identifies that prescription pattern clues, linking automatically to whom that doctor is, his specific office from where the prescription was issued, and its doctor's legal code ID (CRM in Brazil), telephone number, addresses plus other valuable information.

F2.5 The final product of this intelligent cumulative recognition pattern system (fully developed by the author) is to open doors for a fast medical prescription contents capture by the server side, such doctors ID (CRM in Brazil), hospitals ID, health plans/insurance ID and etc., improving statistics in accuracy and speed (all data capturing fully automated from doctor's prescriptions images) without transcriptions based in human interventions subjected to many errors.

| Term | Definition |
|---|---|
| Prescription's Image | It Is an electronic copy of the both |
| | surfaces of the drug(s) prescription |
| | paper form issued by any doctor. |
| Touch Screen Skin | It is a thin transparent surface like a skin |
| | placed over the entire monitor's surface |
| | which contains sensors that captures the touch |
| | either made by a stylist pen or a human finger. |

What is claimed is:

1. It's the new use of Lottery terminals or future assembled-like terminals, from now on known as “DCPS terminal”, in the purpose of capturing pharmaceutical (doctor/consumer/drug/discount coupon) and drug sales information, done at pharmacies, drugstores or like.

2. Digital Capturing Pharmaceutical System—Client Side—Pharmaceutical Capture System running on the DCPS terminal or future assembled like, on pharmacies or drugstores with the special characteristic of being the sole system to run and to use the hardware resources set (motherboard, barcode readers, hard drives, game slip image scanner or like, touch screen, screen, keyboard, mouse, cd-r/rw unit, dvd-r/rw unit, printer, dial-up modems, flash memory devices, tapes, networking and communication ports) in the purpose of capturing electronic information data from sold and non-sold drugs plus the images of the corresponding doctor's prescription (its all types of copies or its hard copy) and discount coupons sourced in the same hardware set. Each fields set plus images are recorded on the DCPS terminal's hard drive, or on-line transmitted, or recorded into any type of existing electronic or optical medias.

3. DCPS Digital Capture Pharmaceutical System—Server Side: This system works by the server side in dependence to DCPS System—Client Side. The claim is the appending mode of information data on database tables, based on the selective learning process of the identifiers from specific doctor's prescription images.

4. DCPS Terminal—Surrounded “in-loco” OCR capture of the Doctor's code ID field image upon the touch screen surface. The scanned prescription full image (front and back images) will be displayed on the DCPS terminal screen. This brand new feature, will allow an exclusive instant OCR and digital capture of the doctor's code ID (CRM in Brazil). In order to achieve it, the operator will touch and drag his finger upon the displayed image on the screen, surrounding directly (forming an imaginary text box around the doctor's code IMAGE) the doctor's code ID (CRM in Brazil), selecting it, for an instant micro OCR resulting as a digital capture of it.

5. (DCPS Terminal) make it possible to capture information about missing medicine on the local pharmacy or drugstore using a magic search on the drugs' terminal table for specifying that missing drug.

6. (DCPS Terminal) Capturing of the out of stock drug information and its substitute information on local pharmacies/drugstores.
